(19) United States
(12) Patent Application Publication
(10) Pub. No.: US 2013/0029989 A1
(43) Pub. Date: Jan. 31, 2013

(54) TOPICAL TREATMENTS FOR PAIN

(71) Applicants: Terence Coderre, Pointe-Claire (CA); Andre Laferriere, Montreal (CA); Vaigunda Ragavendran Jegadeesan, Montreal (CA)

(72) Inventors: Terence Coderre, Pointe-Claire (CA); Andre Laferriere, Montreal (CA); Vaigunda Ragavendran Jegadeesan, Montreal (CA)

(73) Assignee: The Royal Institution for the Advancement of Learning/McGill University, Montreal, QC (CA)

(21) Appl. No.: 13/640,069

(22) PCT Filed: Apr. 13, 2011
(86) PCT No.: PCT/CA2011/000424

§ 371 Date: Oct. 9, 2012

Related U.S. Application Data
(60) Provisional application No. 61/324,348, filed on Apr. 15, 2010.

(51) Int. Cl. A61K 31/522; A61K 31/5377; A61P 25/00

(52) U.S. Cl. 514/236.2; 514/263.32; 514/263.31

(57) ABSTRACT

The present invention relates to novel compositions and therapeutic methods for the treatment of pain, in particular neuropathic, ischemic, muscle, arthritic or multiple sclerosis pain. The compositions include a combination of an alpha2-adrenergic agonist or a nitric oxide donor combined with a phosphodiesterase (PDE) or a phosphatidic acid (PA) inhibitor, which have been found to act together synergistically to provide effective treatment for pain, especially when administered topically.

### FIELD OF THE INVENTION

This invention relates to novel pharmaceutical compositions and methods for treating neuropathic, ischemic and muscle pain. In particular, the present invention relates to topical compositions including a combination of ingredients that provide surprisingly effective relief from pain, and to methods for administration thereof.

### BACKGROUND OF THE INVENTION

Complex regional pain syndrome (CRPS) is a chronic progressive disease characterized by severe pain, swelling and changes in the skin. CRPS patients fall into two subtypes: CRPS-I with no major nerve injury and CRPS-II with injury of a major nerve. Tissue injury, in patients with CRPS, leads to the generation of oxygen free radicals and pro-inflammatory cytokines, which cause microvascular injury, including capillary no-reflow, arteriovenous shunting and lower capillary filtration capacity (Matsumura et al., Scand J Plast Reconstr Surg Hand Surg 1996; 30:133-138; van der Laan et al., Neurology 1998; 51:20-25; Schurmann et al., J Vasc Res 2001; 38:453-461). Evidence using an animal model of CRPS-I, where the animals have chronic post-ischemia pain (CPIP), has indicated that the pain depends on microvascular dysfunction (Coderre et al., Pain 2004; 112:94-105; Xanthos et al., Pain 2008; 137:640-651; Laferriere et al., Mol Pain 2008; 4:49). Thus, animals with CPIP have pain, allodynia, vasospasms, poor tissue perfusion, and oxidative stress. Vasospasms, associated with reduced nitric oxide and increased vasoactive responses to norepinephrine, and capillary no-reflow, lead to reduced nutritive blood flow, poor muscle oxygenation and the build-up of muscle lactate, all of which contribute to the pain (Xanthos et al., Pain 2008; 137:640-651; Laferriere et al., Mol Pain 2008; 4:49). It has been shown that their pain/allodynia and microvascular dysfunction are attenuated by systemic treatments with an alpha₂-adrenergic agonist or nitric oxide donors, which improve thermoregulatory blood flow (relieving vasospasms), as well as by a phosphodiesterase (PDE) inhibitor, which improves nutritive blood flow (relieving capillary no-reflow) (Xanthos et al., Pain 2008; 137:640-651; Laferriére et al., Mol Pain 2008; 4:49; unpublished results). These findings support various observations in CRPS patients, indicating there is microvascular dysfunction and poor tissue oxygenation in CRPS-affected limbs.

It has been known for many years that microvascular dysfunction and resulting oxidative stress contributes to the pain of angina and peripheral arterial disease. Recent parallel studies by other groups suggest that microvascular dysfunction and resulting oxidative stress may contribute to neuropathic pain (including traumatic neuropathy and diabetic neuropathy) and muscle pain (including fibromyalgia and chronic low back pain).

Alpha₂-adrenergic agonists, nitric oxide donors or PDE inhibitors have been used systemically to treat pain associated with angina, peripheral arterial disease, CRPS, neuropathic pain, diabetic neuropathy and chronic low back pain, indicating their usefulness in these syndromes. Phosphatidic acid (PA) inhibitors have not been used to treat pain, but have recently been shown to reduce IL-12/STAT4-induced differentiation of Th1 cells, having beneficial effects in autoimmune Th1-mediated diseases, such as diabetes and experimental allergic encephalitis (an animal model of multiple sclerosis), and may also affect rheumatoid arthritis, which exhibits alterations in Th-1 cell function. Recent evidence also suggests that PA may be an important mediator of demyelination and neuropathic pain following nerve injury. PA inhibitors also have anti-oxidant, anti-cytokine, anti-leukocyte, immunosuppressant and mitochondrial protective effects, in addition to the vasodilator, anti-ischemic and anti-platelet aggregation effects they share with PDE inhibitors.

Most treatments for neuropathic pain, CRPS and ischemia pain currently in use are oral systemic treatments which cause significant side-effects. These side-effects hinder the ability to use therapeutically effective dose levels and reduce patient compliance. There is a need therefore for agents, such as topical agents, which can be used effectively at low doses and which overcome these side effects.

In addition, there are no prescription treatments for pain aimed at increasing tissue oxygenation, which could be used to treat neuropathic, inflammatory, ischemic or muscle pain. It would be desirable therefore to be provided with agents which increase tissue oxygenation for use in treating pain.

### SUMMARY OF THE INVENTION

We report herein the identification of novel combinations of agents and compositions thereof, comprising an alpha₂-adrenergic agonist or a nitric oxide donor and a phosphatidic acid (PA) inhibitor or a phosphodiesterase (PDE) inhibitor. Methods for the prevention and treatment of pain using the combinations and compositions of the invention are also provided herein. In an aspect, the combinations and compositions of the invention are formulated for topical, e.g. transdermal administration.

There are provided herein topical compositions for the treatment of pain comprising a therapeutically effective amount of an alpha₂-adrenergic agonist or a nitric oxide donor and a therapeutically effective amount of a phosphatidic acid (PA) inhibitor or a phosphodiesterase (PDE) inhibitor, formulated in a pharmaceutically acceptable carrier for a topical composition. In some embodiments, the compositions of the invention may comprise an alpha₂-adrenergic agonist and a PA or a PDE inhibitor; a nitric oxide donor and a PA or a PDE inhibitor; or an alpha₂-adrenergic agonist and/or a nitric oxide donor and a PA inhibitor and/or a PDE inhibitor.

In an embodiment, the alpha₂-adrenergic agonist used in the combinations and compositions of the invention is apraclonidine, clonidine, detomidine, dexamedetomidine, guanabenz, guanfacine, moxonidine, romifidine, tizanidine or xylazine. In another embodiment, the nitric oxide donor used in the combinations and compositions of the invention is isosorbide dinitrate, L-arginine, linsidomine, minoxidil, nicorandil, nitroglycerin, nitroprusside, nitrosoglutathione, or S-nitroso-N-acetyl-penicillamine (SNAP). In yet another embodiment, the PA inhibitor used in the combinations and compositions of the invention is lisofylline or pentoxifylline. In a further embodiment, the PDE inhibitor used in the combinations and compositions of the invention is acetildenafil, avanafil, bucladesine, cilostamide, cilostazol, dipyridamole, enoximone, glaucine, ibudilast, icariin, inamrinone (formerly amrinone), lodenafil, luteolin, milrinone, mirodenafil, pentoxifylline, piclamilast, pimobendan, propentofylline, roflumilast, rolipram, RPL-554, sildenafil, tadalafil, udenafil, vardenafil or zardaverine.

In certain embodiments, the composition comprises clonidine and pentoxifylline; linsidomine and pentoxifylline; apraclonidine and lisofylline; linsidomine and lisofylline; or SNAP and lisofylline.

The combinations and compositions of the invention may also include additional ingredients which increase the analgesic effectiveness of the combinations and compositions. For example, additional ingredients which increase penetration of the alpha₂-adrenergic agonist, nitric oxide donor, PA inhibitor and/or PDE inhibitor may be included in the combinations and compositions of the invention. Non-limiting examples of such additional ingredients include analgesics such as cyclooxygenase inhibitors, NSAIDs, NMDA receptor antagonists, tricyclic antidepressants, α2δ calcium channel agents and guanethidine.

The topical compositions may be incorporated into formulations for topical, e.g. transdermal administration, of which many are known in the art. Non-limiting examples of such formulations include creams, lotions, gels, oils, ointments, sprays, foams, liniments, aerosols and transdermal devices for absorption through the skin.

In an embodiment, the combinations and compositions of the invention include about 0.005-0.5% W/W of apraclonidine, about 0.0075-0.1% WAN of clonidine or about 0.2-2% W/W of linsidomine, in combination with about 0.0078-0.5% W/W of lisofylline or about 0.075-5% W/W of pentoxifylline. In other embodiments, the amount of apraclonidine in the composition is equal to or less than 0.5% WAN, the amount of clonidine in the composition is equal to or less than 0.1% W/W, the amount of lisofylline in the composition is equal to or less than 0.5% W/W, the amount of pentoxifylline in the composition is equal to or less than 5% W/W, and/or the amount of linsidomine in the composition is equal to or less than 2% WAN.

Methods for preventing or treating pain comprising topical administration of the combinations and compositions of the invention are also provided herein. In an embodiment, the pain is neuropathic, ischemic or muscle pain. In other embodiments, the pain may be associated with diabetic neuropathy, complex regional pain syndrome (CRPS), angina, peripheral arterial disease, arthritis, inflammation, multiple sclerosis, fibromyalgia, or chronic low back pain. In yet other embodiments, methods and compositions for the treatment or prevention of neuropathy, e.g. peripheral neuropathy, ischemic pain, chronic muscular pain, and/or complex regional pain syndrome (CRPS) are provided herein.

In one embodiment, methods and compositions are provided for the treatment of peripheral neuropathy, comprising a therapeutically effective amount of an alpha₂-adrenergic agonist or a nitric oxide donor and a therapeutically effective amount of a phosphatidic acid (PA) inhibitor or a phosphodiesterase (PDE) inhibitor, formulated in a pharmaceutically acceptable carrier for a topical composition, wherein the alpha₂-adrenergic agonist is apraclonidine in an amount equal to or less than 0.5%, or clonidine in an amount equal to or less than 0.1%; the nitric oxide donor is linsidomine in an amount equal to or less than 2%; the PA inhibitor is lisofylline in an amount equal to or less than 0.5%; and the PDE inhibitor is pentoxifylline in an amount equal to or less than 5%.

In some embodiments, the methods and compositions of the invention increase tissue oxygenation in a subject; increase thermoregulatory and/or nutritive blood flow in a subject; have anti-oxidant, anti-cytokine, immunosuppressant and/or mitochondrial protective effects in a subject; reduce arterial vasospasms and/or capillary no-reflow in a subject; and/or have an anti-allodynic effect.

Pharmaceutical compositions comprising an alpha₂-adrenergic agonist or a nitric oxide donor and a phosphatidic acid (PA) inhibitor or a phosphodiesterase (PDE) inhibitor, and a pharmaceutically acceptable carrier, are also provided herein, as well as pharmaceutical compositions for treating pain comprising the combinations of agents of the invention and a pharmaceutically acceptable carrier. Such pharmaceutical compositions may comprise, for example, clonidine and pentoxifylline; linsidomine and pentoxifylline; apraclonidine and lisofylline; linsidomine and lisofylline; or SNAP and lisofylline; and a pharmaceutically acceptable carrier. In some embodiments, the pharmaceutical compositions may include additional ingredients which increase the analgesic effectiveness of the composition. In other embodiments, the pharmaceutical compositions are adapted for topical, e.g. transdermal administration.

### BRIEF DESCRIPTION OF THE DRAWINGS

Having thus generally described the nature of the invention, reference will now be made to the accompanying drawings, showing by way of illustration, an embodiment or embodiments thereof, and in which:

FIG. 1 shows the effects of ipsilateral hind paw topical application of pentoxifylline (A), clonidine (B), linsidomine (C), lisofylline (D), SNAP (E) or apraclonidine (F) on paw withdrawal threshold (PWT, g) to von Frey hair stimulation in 2-14 day CPIP rats. Each treatment produces anti-allodynic effects as illustrated by significant increases in PWT at the higher doses (*p<0.05, Post Drug vs. Pre Drug);

FIG. 2 shows the effects of ipsilateral hind paw topical application of combinations of a single dose of clonidine (A,B) or linsidomine (C,D) with pentoxifylline on paw withdrawal threshold (PWT, g) to von Frey hair stimulation in 2-14 day CPIP rats (A,C) or on the anti-allodynic (ΔPWT) dose response curve for pentoxifylline (B,D). Clonidine or linsidomine combinations with pentoxifylline both produce significant anti-allodynic effects (A,C) and shift the anti-allodynic dose response curve of pentoxifylline to the left (B,D) (* p<0.05, Post Drug vs. Pre Drug; ‡p<0.05, Post Drug vs. Vehicle);

FIG. 3 shows the effects of ipsilateral hind paw topical application of combinations of a single dose of linsidomine (A,B), apraclonidine (C,D) or SNAP (E,F) with lisofylline on paw withdrawal threshold (PWT, g) to von Frey hair stimulation in 2-14 day CPIP rats (A,C,E) or on the anti-allodynic (□PVVT) dose response curve for lisofylline (B,D,F). Either linsidomine, apraclonidine or SNAP combinations with lisofylline produce significant anti-allodynic effects (A,C,E) and shift the anti-allodynic dose response curve of lisofylline to the left (B,D,F) (* p<0.05, Post Drug vs. Pre Drug);

FIG. 4 shows the effects of ipsilateral vs. contralateral hind paw topical application of combinations of clonidine+pentoxifylline (A), linsidomine+pentoxifylline (B), apraclonidine+lisofylline (C), linsidomine+lisofylline (D), or SNAP+lisofylline (E) on paw withdrawal threshold (PWT, g) to von Frey hair stimulation in 2-14 day CPIP rats. No combination produced significant anti-allodynic effects when applied to the contralateral hind paw, despite significant effects when applied to the ipsilateral hind paw (* p<0.05, Post Drug vs. Pre Drug; t p<0.05, ipsilateral Post Drug vs. contralateral Post Drug; ‡ p<0.05, ipsilateral Post Drug vs. ipsilateral Post Vehicle);

FIG. 5 shows the effects of ipsilateral vs. contralateral hind paw topical application of combinations of clonidine+pentoxifylline (A), linsidomine+pentoxifylline (B), apraclonidine+lisofylline (C), or SNAP+lisofylline (D) on paw withdrawal threshold (PWT, g) to von Frey hair stimulation in rats with a chronic constriction injury (CCI) of the sciatic nerve 7-14 days earlier (i.e., neuropathic rats). No combination produced significant anti-allodynic effects when applied to the contralateral hind paw, despite significant effects when applied to the ipsilateral hind paw (* p<0.05, Post Drug vs. Pre Drug; t p<0.05, ipsilateral Post Drug vs. contralateral Post Drug; ‡ p<0.05, ipsilateral Post Drug vs. ipsilateral Post Vehicle);

FIG. 6 shows the effects of the combination of apraclonidine+lisofylline on the duration of responses to acetone (cold allodynia) in 10-month-old SPARC-null mice with significant degenerative disc disease. The apraclonidine+lisofylline, but not vehicle, ointment significantly reduced cold allodynia 45 min after application to the hind paw (* p<0.05, Post Drug vs. Pre Drug; ‡ p<0.05, Post Drug vs. Vehicle);

FIG. 7 shows the effects of the combination of linsidomine+pentoxifylline on withdrawal threshold (PWT, g) to von Frey hair stimulation of the hind paw in rats with referred muscle pain produced by 2 injections of 100 μl of acidic saline (pH 4.0) into the gastrocnemius muscle over 5 days, with testing 24 h after the second injection. Combining 0.4% linsidomine with 0.4%, but not 0.15%, pentoxifylline significantly reduced mechanical allodynia 60 min after application to the hind paw (* p<0.05, Post Drug vs. Pre Drug);

FIG. 8 shows the effects of the combination of linsidomine+pentoxifylline on withdrawal threshold (PWT, g) to von Frey hair stimulation of the hind paw in rats with inflammatory pain induced by plantar hind paw injection of 50 μl of 1 mg/ml complete Freund's adjuvant (CFA) 48 h before testing. Combining 0.4% linsidomine with 0.4%, but not 0.15%, pentoxifylline significant reduced mechanical allodynia 45 min after application to the hind paw (* p<0.05, Post Drug vs. Pre Drug);

FIG. 9 A) shows Laser Doppler flux measurements (in arbitrary units—AU) illustrating the effects of 25 mg/kg systemic pentoxifylline on post-occlusive reactive hyperemia in a sham rat (upper black trace) and a 2 day CPIP rat (lower grey trace). The CPIP rat displayed markedly lower post-occlusive reactive hyperemia (evidence of microvascular dysfunction) after the initial period of ischemia compared to the sham animal, but a noticeably elevated response during the second reperfusion period, post-PTX administration. B) shows the mean Laser Doppler flux measures (as log of the % difference to pre-ischemia baseline) during the rapid post-occlusive reactive hyperemic period (first 100 sec after reperfusion) for sham rats and day 2-8 CPIP rats. Asterisks (*p<0.05) indicate the presence of a significant difference between pre- and post-drug measures at the same time-points post-reperfusion. The reduced post-occlusive reactive hyperemia in CPIP rats is significant reversed by the pentoxifylline treatment, which has no significant effect in sham rats. C) shows that both 25 and 50 mg/kg of pentoxifylline produce anti-allodynic effects in CPIP rats elevating PWTs to von Frey hair stimulation (*p<0.05 compared to vehicle); and

FIG. 10 shows the tissue oxygenation index (TOI) recordings using near infrared spectroscopy (NIRS) in the affected limb of two CRPS-I patients (closed circles) and in contralateral or healthy control limbs (open circles) before, during and after exercise or ischemia. A) shows Palmar TOI before exercise, during exercise and post-exercise in a CRPS patient and a gender-matched control subject. Basal TOI and TOI during exercise is lower in the CRPS-I affected hand than in a healthy subject control hand. After exercise TOI is elevated reflecting an abnormal hyperoxygenation in the CRPS-I hand. B) shows basal forearm TOI and TOI during ischemia is lower in the affected CRPS-I arm than the contralateral arm. Reactive hyperoxygenation is also delayed in the CRPS-I arm, reflecting microvascular dysfunction.

### DETAILED DESCRIPTION

There are provided herein novel topical combinations of agents for the pharmacologic treatment of pain, including without limitation neuropathic pain, ischemic pain and muscle pain. The combinations of agents provided herein comprise combinations of alpha₂-adrenergic agonists or nitric oxide donors with phosphatidic acid (PA) or phosphodiesterase (PDE) inhibitors.

The combinations provided herein are based, at least in part, on the novel finding that alpha₂-adrenergic agonists or nitric oxide donors in combination with PA inhibitors or PDE inhibitors produce synergistic effects when used in combination. While alpha₂-adrenergic agonists, nitric oxide donors, and PDE inhibitors have been used previously to alleviate chronic pain, they have not been used in combination for the treatment of chronic pain, either systemically or in topical preparations. PDE inhibitors have not been used previously to alleviate pain in topical preparations. Moreover, PA inhibitors have not been used previously to treat chronic pain, even though they are excellent candidates for pain therapeutics due to their pharmacological properties, such as vasodilator, anti-ischemic, and anti-platelet aggregation effects.

We report herein for the first time the surprising and unexpected finding that alpha₂-adrenergic agonists or nitric oxide donors in combination with PA inhibitors or PDE inhibitors act synergistically in the treatment of pain. The combinations provided herein have not been used previously for pain therapy and the very high degree of synergy obtained using the alpha₂-adrenergic agonists or nitric oxide donors combined with PA inhibitors or PDE inhibitors was unexpected. Our finding is based, at least in part, on our discovery that neuropathic pain and CRPS depend in part on regional microvascular dysfunction that could be alleviated by enhancing local thermoregulatory and nutritive blood flow. These mechanisms are not widely recognized as being involved in pain, and have only been recently attributed to neuropathic pain and CRPS. Microvascular dysfunction and poor tissue perfusion have also only recently been considered as contributing to fibromyalgia and chronic low back pain. Without wishing to be bound by theory or by a particular mechanism, it is believed that the combinations are beneficial, at least in part, because of their actions on both microvascular dysfunction (vasospasms) and injury (capillary no-reflow).

The present invention is thus based, at least in part, and again without wishing to be limited by theory, on the discovery that treatments aimed at enhancing tissue oxygenation, by for example reducing arterial vasospasms and capillary no-reflow, will effectively relieve pain. We have exploited the novel theory that improving both thermoregulatory and nutritive blood flow together will act synergistically to produce improved tissue oxygenation that will reduce pain, including for example neuropathic, ischemic, inflammatory and muscle pain.

Thus in one embodiment, the combinations provided herein increase tissue oxygenation. In another embodiment, the combinations provided herein increase thermoregulatory and/or nutritive blood flow. The combinations of the invention may also produce anti-oxidant, anti-cytokine, immunosuppressant and/or mitochondrial protective effects, which contribute to pain in many syndromes. In an embodiment, the combinations provided herein reduce arterial vasospasms and/or capillary no-reflow. In another embodiment, microvascular dysfunction is treated by the combinations provided herein. In yet another embodiment, the combinations provided herein have an anti-allodynic effect.

In addition, the topical administration of the combinations of the invention reduces potential systemic side-effects. Although single agents have previously been used for neuropathic pain (including diabetic neuropathy), angina and peripheral arterial disease (including intermittent claudication and chronic limb ischemia), CRPS, chronic low back pain and fibromyalgia, topical treatment has not often been considered. In addition, as mentioned above the specific combinations presented herein have not been used for pain therapy previously. Thus in one aspect, the combined effects of the synergy between the agents, which allows for lower doses to be used, and the topical application of the agents, combinations or compositions, has the significant advantage of reducing side effects while maintaining high potency.

Accordingly, we provide herein topical treatments for neuropathic pain (such as diabetic neuropathy and CRPS-II), ischemic pain (such as angina, CRPS-I and peripheral arterial disease), muscle pain (such as fibromyalgia and chronic low back pain), arthritic or inflammatory pain and MS pain based on synergistic combinations of alpha₂-adrenergic agonists or nitric oxide donors with PA or PDE inhibitors. The combinations may produce synergistic effects by increasing both local thermoregulatory and nutritive blood flow, as well as producing anti-oxidant, anti-cytokine, immunosuppressant and/or mitochondrial protective effects, which contribute to pain in these syndromes. As topical agents, producing only local effects, these treatments should have no or minimal central nervous system side-effects, or abuse potential, as occurs with many standard therapies.

Both alpha₂-adrenergic agonists and nitric oxide donors are agents that act peripherally to reduce arterial vasospasms; alpha₂-adrenergic agonists by inhibiting the local release of vasoconstrictive norepinephrine after binding to alpha₂-adrenergic receptors on sympathetic post-ganglionic neurons (in addition to central actions), and nitric oxide donors by increasing the production of the potent vasodilator nitric oxide. PA inhibitors prevent the generation of PA by blocking lysophosphatidic acid acyltransferase (LPAAT), which catalyzes the acylation of lysophosphatidic acid (LPA) to PA. PA is a key messenger in a common signaling pathway activated by proinflammatory mediators such as interleukin-1β, tumor necrosis factor α and platelet activating factor. PDE inhibitors relax blood vessels by inhibiting phosphodiesterases, which degrade the intracellular second messengers cyclic adenosine monophosphate (cAMP) and cyclic guanosine monophosphate (cGMP).

In an embodiment, the combination of agents comprises a combination of an alpha₂-adrenergic agonist and a PA inhibitor. In another embodiment, the combination of agents comprises a combination of an alpha₂-adrenergic agonist and a PDE inhibitor. In yet another embodiment, the combination of agents comprises a combination of a nitric oxide donor and a PA inhibitor. In a further embodiment, the combination of agents comprises a combination of a nitric oxide donor and a PDE inhibitor.

In one embodiment, the combination of agents comprises an alpha₂-adrenergic agonist and a nitric oxide donor in combination with a PA inhibitor. In another embodiment, the combination of agents comprises an alpha₂-adrenergic agonist and a nitric oxide donor in combination with a PDE inhibitor. In yet another embodiment, the combination of agents comprises an alpha₂-adrenergic agonist or a nitric oxide donor in combination with a PA inhibitor and a PDE inhibitor. In a further embodiment, the agent comprises an alpha₂-adrenergic agonist and a nitric oxide donor in combination with a PA inhibitor and a PDE inhibitor.

Exemplary alpha₂-adrenergic agonists for use in the combinations of the invention include, without limitation, apraclonidine, clonidine, detomidine, dexamedetomidine, guanabenz, guanfacine, moxonidine, romifidine, tizanidine and xylazine. In a particular embodiment, apraclonidine is the preferred agent.

Exemplary nitric oxide donors for use in the combinations of the invention include, without limitation, isosorbide dinitrate, L-arginine, linsidomine, minoxidil, nicorandil, nitroglycerin, nitroprusside, nitrosoglutathione, and S-nitroso-N-acetyl-penicillamine (SNAP). In a particular embodiment, linsidomine is the preferred agent.

Exemplary PA inhibitors for use in the combinations of the invention include, without limitation, lisofylline and pentoxifylline (of which lisofylline is a metabolite). In a particular embodiment, lisofylline is the preferred agent.

Exemplary PDE inhibitors for use in the combinations of the invention include, without limitation, PDE3 inhibitors, such as bucladesine, cilostamide, cilostazol, enoximone, inamrinone (formerly amrinone), milrinone, pimobendan and zardaverine; PDE4 inhibitors such as glaucine, ibudilast, luteolin, pentoxifylline, piclamilast, propentofylline, roflumilast, rolipram and RPL-554; and PDE5 inhibitors, such as acetildenafil, avanafil, dipyridamole, icariin, lodenafil, mirodenafil, sildenafil, tadalafil, udenafil, and vardenafil. In a particular embodiment, the PDE inhibitor is a PDE4 inhibitor.

Exemplary combinations of the invention include the following: a combination comprising apraclonidine and lisofylline; a combination comprising linsidomine and lisofylline; a combination comprising SNAP and lisofylline; a combination comprising clonidine and pentoxifylline; and a combination comprising linsidomine and pentoxifylline. In a particular embodiment, the combination of the invention comprises apraclonidine and lisofylline.

It is contemplated that other alpha₂-adrenergic agonists, nitric oxide donors, PA inhibitors and PDE inhibitors known in the art may be used in the combinations of the invention.

As used herein the term “synergistic” refers to a pain-reducing or pain-treating response elicited through the synergistic effect of the agents described herein, in which the combined effect of multiple agents is greater (in duration, intensity, comprehensively or otherwise) than the sum of the effect produced by each agent alone.

As used herein the terms “topical” or “transdermal” delivery are used interchangeably to refer to delivery of a drug by passage into and through the skin or mucosal tissue.

### Methods of Treatment and Medical Uses

The therapeutic agents provided herein (including for example combinations, compositions and topical treatments of the invention) can be used to provide effective, long-term relief from the pain of peripheral neuropathies, such as CRPS-II and diabetic neuropathy, from the pain of ischemic conditions, such as angina, CRPS-I and peripheral arterial disease, from chronic muscle pain such as fibromyalgia and chronic low back pain, from arthritic pain, from the pain of multiple sclerosis (MS), and from other related or similar pain disorders.

Peripheral neuropathy is a condition involving nerve-end damage anywhere in the body. Peripheral neuropathy generally refers to a disorder that affects the peripheral nerves, most often manifested as one or a combination of motor, sensory, sensorimotor, or autonomic neural dysfunction. The wide variety of morphologies exhibited by peripheral neuropathies can each be uniquely attributed to an equally wide variety of causes. For instance, peripheral neuropathies can be genetically acquired, can result from a systemic disease, can manifest as a post-surgical complication, or can be induced by a toxic agent. Some toxic agents that cause neurotoxicities are therapeutic drugs, antineoplastic agents, contaminants in foods or medicinals, and environmental and industrial pollutants. In other cases, neuropathy may be due to low back pain, Guillain-Barre Syndrome, or sciatica.

Although a number of neuropathies are related to the disease diabetes mellitus, others, although not known to be related to diabetes, are similar in their physiological effects on the peripheral vascular system. Such diseases include Raynaud's Phenomenon, including CREST syndrome, autoimmune diseases such as erythematosus, and rheumatoid diseases. Other peripheral neuropathies include the following: HIV-associated neuropathy; B12-deficiency associated neuropathy; cranial nerve palsies; drug-induced neuropathy; industrial neuropathy; lymphomatous neuropathy; myelomatous neuropathy; multi-focal motor neuropathy; chronic idiopathic sensory neuropathy; carcinomatous neuropathy; acute pain autonomic neuropathy; alcoholic neuropathy; compressive neuropathy; vasculitic/ischemic neuropathy; and mono- and poly-neuropathies.

For example, among the most important toxic agents causing peripheral neuropathy are therapeutic agents, particularly those used for the treatment of neoplastic disease. In certain cases, peripheral neuropathy is a major complication of cancer treatment and is the main factor limiting the dosage of chemotherapeutic agents that can be administered to a patient. Peripheral neuropathies can also contribute to other pain syndromes including chronic low back pain, fibromyalgia, CRPS-II and phantom limb pain.

The therapeutic agents of the invention may be used for many types of pain, including fibromyalgia, chronic wide spread pain, and pain which may depend on nerve and/or ischemic injury. Chronic angina, peripheral arterial disease, and arthritic pain are also encompassed, as well as other pain disorders known in the art.

Multiple sclerosis (MS) is a disease in which the fatty myelin sheaths around the axons of the brain and spinal cord neurons are damaged, leading to demyelination and scarring as well as a broad spectrum of signs and symptoms. The majority of patients with MS experience acute or chronic pain. For example, MS patients may experience chronic pain syndromes such as dysesthesia, low back pain, spasms, tonic seizures, tightening and painful sensations in the extremities. Acute pain syndromes may include neuralgia, L'Hermitte's sign and pain associated with optic neuritis. For many, pain is one of the most severe symptoms of MS.

Arthritis refers to a group of conditions involving damage to the joints of the body. There are over 100 different forms of arthritis. The most common form, osteoarthritis (degenerative joint disease) is a result of trauma to the joint, infection of the joint, or age. Other arthritis forms are rheumatoid arthritis, psoriatic arthritis, and autoimmune diseases in which the body attacks itself. Septic arthritis is caused by joint infection. The major complaint by individuals who have arthritis is pain, which is often a constant and daily feature of the disease. The pain may be localized to the back, neck, hip, knee or feet and may occur due to inflammation that occurs around the joint, damage to the joint from disease, daily wear and tear of joint, muscle strains caused by forceful movements against stiff, painful joints and fatigue.

Complex regional pain syndrome (CRPS) is a chronic progressive disease characterized by severe pain, swelling and changes in the skin. In patients with CRPS, tissue injury leads to the generation of oxygen free radicals and pro-inflammatory cytokines, which cause microvascular injury, including arterial vasospasms and capillary no-reflow in the blood vessels of muscle and nerve. Vasospasms, associated with reduced nitric oxide and increased vasoactive responses to norepinephrine, and capillary no-reflow, lead to reduced nutritive blood flow, poor muscle oxygenation and the build-up of muscle lactate, all of which contribute to the pain. CRPS is divided into two types: Type I, formerly known as reflex sympathetic dystrophy (RSD), Sudeck's atrophy, reflex neurovascular dystrophy (RND) or algoneurodystrophy, and Type II, formerly known as causalgia, which is distinguished from CRPS-I by the presence of an accompanying major nerve injury. The cause of this syndrome is currently unknown but precipitating factors include injury and surgery.

Ischemic pain is associated with decreased blood flow caused by mechanical obstruction, constricting orthopedic casts, or insufficient blood flow that results from injury or surgical trauma. Ischemic pain caused by occlusive arterial disease, such as an embolus or thrombus, is often severe and may not be relieved, even with narcotics. Ischemic pain traditionally includes pain associated with coronary (angina) or peripheral (intermittent claudication, critical limb ischemia) arterial disease. Many conditions such as peripheral vascular disease and partial arterial occlusion can lead to ischemic pain.

Accordingly, combinations of agents and compositions thereof of the invention may be used in the treatment or prevention of pain. It is contemplated that the combinations and compositions of the invention are used in the treatment or prevention of the types of pain discussed herein as well as other pain disorders which are known in the art.

The combinations of agents and compositions of the invention are indicated both in the therapeutic and/or prophylactic treatment of the diseases and conditions discussed herein. Accordingly, in an aspect of the present invention, there is provided a method of treatment or prevention of peripheral neuropathy, or of a disorder characterized by peripheral neuropathy, comprising administration of a combination of agents or composition thereof of the invention to a subject. In an aspect, methods and compositions for the topical or transdermal treatment of neuropathy are provided herein. More particularly, transdermal or topical compositions including a combination of ingredients that provide a surprising degree of effective relief from the symptoms of peripheral neuropathy and methods for administering the compositions to treat various neuropathies are provided herein.

In accordance with another aspect, there is provided a method of treatment or prevention of arthritic pain, comprising administration of a combination of agents or composition of the invention to a subject. According to another aspect of the present invention, there is provided a method of treatment or prevention of CRPS, comprising administration of a combination of agents or composition of the invention to a subject. According to yet another aspect of the present invention, there is provided a method of treatment or prevention of ischemic pain, comprising administration of a combination of agents or composition of the invention to a subject. According to a further aspect of the present invention, there is provided a method of treatment or prevention of MS pain, comprising administration of a combination of agents or composition of the invention to a subject. In another aspect, pain syndromes associated with chronic muscle pain are treated or prevented by administration of a combination of agents or compositions thereof disclosed herein to a subject.

In other aspects, there are provided methods directed to treating pain, comprising the step of transdermal or topical administration of an effective amount of a pharmaceutical composition of a combination of agents of the invention to the affected area of a subject in need of such treatment. Other drugs or ingredients may be added as needed to increase the analgesic effect.

Further embodiments include methods for treating pain in a subject, the methods comprising topically administering an effective amount of a composition comprising an alpha₂-adrenergic agonist or a nitric oxide donor combined with a therapeutically effective amount of a PA or a PDE inhibitor, formulated in a pharmaceutically acceptable topical carrier.

Other embodiments include methods for treating a subject suffering from neuropathic pain, the method comprising topically administering an effective amount of a composition comprising an alpha₂-adrenergic agonist or a nitric oxide donor combined with a therapeutically effective amount of a PA or PDE inhibitor, formulated in a pharmaceutically acceptable carrier for topical treatment. In an embodiment, the neuropathic pain is peripheral neuropathic pain.

It should be understood that, in addition to preventing or treating pain, combinations of agents and compositions thereof of the invention may increase tissue oxygenation; increase thermoregulatory and/or nutritive blood flow; have anti-oxidant, anti-cytokine, immunosuppressant and/or mitochondrial protective effects; reduce arterial vasospasms and/or capillary no-reflow; and/or have an anti-allodynic effect in a subject being treated.

As used herein, “subject” includes mammals, including humans.

In an embodiment, the methods disclosed herein comprise administration of a therapeutically effective amount of a combination of agents or a composition of the invention, to a subject in need thereof. A subject “in need thereof” is a subject suffering from or susceptible to the pain disorder or condition in question. The term “therapeutically effective amount” refers to an amount of a compound, which confers a therapeutic effect on the treated subject. The effect may be objective (i.e. measurable by some test or marker) or subjective (i.e. the subject gives an indication of or feels an effect). The term “effective amount” refers to an amount of a compound, combination or composition, which is sufficient to produce the desired result or has the desired biological effect. For example, an effective amount may be an amount, which at least partly alleviates, reduces, prevents or treats pain in a subject.

Use of the combinations of agents of the invention in the manufacture of a medicament for treating pain and the disorders disclosed herein are also encompassed, as are compositions for use for treating or preventing the described pain disorders and conditions.

### Pharmaceutical Compositions

Most treatments for neuropathic pain, CRPS and ischemic pain use oral systemic treatments, which cause significant side-effects. These side-effects hinder the ability to use therapeutically effective dose levels and reduce patient compliance. Use of topical agents in low doses can overcome these side-effects. With topical treatment, drug concentrations are higher at local target sites, but plasma concentrations will typically be less than 10% of the same dose given orally. Furthermore, topical treatment avoids gastrointestinal (GI) tract and hepatic first pass metabolism, and allows more drug to be active locally with less potential liver or GI toxicity.

We report herein that topical administration of combinations of agents of the invention in our animal model of CRPS-I produced effects similar to those produced by systemic administration of the individual agents alone at 5 to 200 times higher systemic doses. Moreover, in the same model, administration of the topical combinations had an efficacy greater than systemic acetaminophen, ibuprofen, dexamethasone or amitriptyline at higher doses (Millecamps and Coderre, Eur J Pharmacol 2008; 583:97-102). The topical combinations also produced maximal effects in both animal models of CRPS-I and neuropathic pain that are equivalent to those produced by high systemic doses of morphine and pregabalin (the gold standard treatment for neuropathic pain) (Millecamps and Coderre, Eur J Pharmacol 2008; 583:97-102; Kumar N et al., J. Neurochem. 2010; 113: 552-61). It is noted that these results were achieved using concentrations of topical agents in the combinations, which are much lower than the recommended concentrations used for neuropathic or ischemic pain or clinical uses. For example, apraclonidine at 0.005%, clonidine at 0.0075%, lisofylline at 0.0078%-0.075%, pentoxifylline at 0.3 or 0.6%, and linsidomine at 0.4% were all found to be highly effective when administered topically in the combinations of the invention to the animals (see Examples). In contrast, the typical recommended concentrations for these agents when used alone are apraclonidine at 0.5-1.0%, clonidine at 0.1-0.3%, lisofylline at 0.5-5%, pentoxifylline at 5-15% and linsidomine at 2%. Thus, the synergy produced by combining these agents results in significant anti-allodynic effects at doses that are 5 to 640 times lower than the topical doses that are used for the single agents.

Acceptable dose ranges which could be used for each of the agents in the combinations and compositions of the invention include: apraclonidine at 0.005-0.5%, clonidine at 0.007-0.1%, lisofylline at 0.063-0.50%, pentoxifylline at 0.075-5%, and linsidomine at 0.2-2%. In other embodiments, the range of apraclonidine used is 0.005-0.04%, the range of clonidine used is 0.007-0.06%, the range of lisofylline used is 0.063-0.25% or the range of linsidomine used is 0.2-1.6%. These ranges are intended to be exemplary and should not be taken as limiting the invention.

In accordance with one embodiment of the present invention, there are provided pharmaceutical compositions and formulations of combinations of agents of the invention. In an embodiment, there are provided herein topical or transdermal compositions and formulations of the combinations of agents of the invention.

Suitable topical formulations of the combinations and compositions of agents of the invention include without limitation transdermal devices, aerosols, gels, creams, ointments, lotions, liniments, dusting powders, patches, hydrogel patches, and the like.

It is well known in the art that therapeutic agents can be formulated in a pharmaceutically acceptable diluent or carrier suitable for topical or transdermal use. Except insofar as any conventional medium or agent is incompatible with the active ingredients, use thereof in the pharmaceutical compositions described herein is contemplated. Supplementary active ingredients can also be incorporated into the compositions. For example, the topical composition may additionally include another agent with analgesic properties or another agent treating the underlying cause of the pain.

The topical preparations and compositions provided herein include any formulations suitable for topical or transdermal application, and include without limitation: aqueous creams, ointments, gels, lotions, roll-on liquids, sprays, glass bead wound dressings, and synthetic polymer dressings impregnated with the compositions described herein. These preparations may also include compounds, such as dimethylsulfoxide, which would facilitate the passage of the active ingredients across the skin keratin barrier and into the epidermis. In one embodiment, the preparation is a cream. For other formulations, the combinations of the invention can also be incorporated into oils, foams, liniments, aerosols or transdermal devices for absorption through the skin. In one embodiment, formulations or means of administration which result in systemic administration are excluded, in order to avoid side effects.

The compositions described herein can be administered in therapeutically effective amounts. A therapeutically effective amount means the amount required to at least partly attain the desired effect, i.e., to alleviate, reduce, treat or prevent the pain.

Such amounts will depend, of course, on the particular condition being treated, the nature or severity of the condition, and individual patient parameters. These include age, physical condition, size, weight and other concurrent treatment. The magnitude of prophylactic or therapeutic dose of a combination of agents or a composition of the invention will also vary with the particular combination or composition of the invention and its site or route of administration. The optimal dosage will be determined by the skilled person using art-recognized techniques and the amounts prescribed will be at the discretion of the attending physician. These factors are well known to those of ordinary skill in the art, and can be addressed with no more than routine experimentation. It is generally preferred that a minimum effective dose be determined according to sound medical judgment. It will be understood by those of ordinary skill in the art, however, that a higher dose may be administered for medical, psychological or other reasons.

The compositions described herein may be applied to the affected area of the skin of the patient. The frequency of application will depend on individual patient circumstances. For example, the compositions may be applied daily, twice daily, or even more frequently.

Methods and pharmaceutical carriers for preparation of pharmaceutical compositions, including compositions for topical administration are well known in the art, as set out in textbooks such as Remington's Pharmaceutical Sciences, 17th Edition, Mack Publishing Company, Easton, Pa., USA (updated in Gennaro, A. R. (Ed.), Remington: The Science and Practice of Pharmacy, 20^{th} edition, Lippincott, Williams & Wilkins) which is incorporated by reference in its entirety.

It should be understood that for the present invention, a topical or transdermal route of administration is generally preferred for providing a mammal, especially a human with an effective dosage of a combination of agents or composition of the invention, in order to avoid side effects which may arise from systemic administration of the agents. Dosage forms may include dispersions, suspensions, solutions, creams, ointments, aerosols, and the like. The most suitable route of administration in any given case will depend on the nature and severity of the condition being treated and on the nature of the active ingredients. They may be conveniently presented in unit dosage forms and prepared by any of the methods well known in the art of pharmacy.

In practical use, the combinations of agents of the invention can be combined as the active ingredient in intimate admixture with a pharmaceutical carrier according to conventional pharmaceutical compounding techniques. The carrier may take a wide variety of forms depending on the form of preparation desired for administration, e.g., topical. Such compositions may be prepared by any of the methods of pharmacy but all methods include the step of bringing into association the active ingredients with the carrier, which constitutes one or more necessary ingredients. In general, the compositions are prepared by uniformly and intimately admixing the active ingredients with pharmaceutically acceptable carriers or diluents.

The pharmaceutical compositions of the present invention comprise combinations of agents described herein, e.g. an alpha₂-adrenergic agonist or a nitric oxide donor combined with a PA inhibitor or a PDE inhibitor, or pharmaceutically acceptable salts thereof, as active ingredients, and may also contain a pharmaceutically acceptable carrier or diluent. The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary, and tertiary amines, substituted amines including naturally occurring substituted amines, cyclic amines and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

It should be understood that references to the agents, combinations, compositions and methods of the invention described herein are meant to also include the pharmaceutically acceptable salts as well as acidic and basic forms of the active ingredients.

It should also be understood that the compositions of the invention may include additional ingredients such as other carriers, moisturizers, oils, fats, waxes, surfactants, thickening agents, antioxidants, viscosity stabilizers, chelating agents, buffers, preservatives, perfumes, dyestuffs, lower alkanols, humectants, emollients, dispersants, sunscreens such as radiation blocking compounds or particularly UV-blockers, antibacterials, antifungals, disinfectants, vitamins, or antibiotics, as well as other suitable materials that do not have a significant adverse effect on the activity of the topical composition. Additional ingredients for inclusion in the carrier are sodium acid phosphate moisturizer, witch hazel extract carrier, glycerin humectant, apricot kernel oil emollient, corn oil dispersant, and the like. Those of skill in the art will readily recognize additional ingredients, which can be admixed in the compositions described herein. The pharmaceutical preparation may also contain non-toxic auxiliary substances such as emulsifying, preserving, wetting, and bodying agents, as for example, polyethylene glycols; antibacterial components such as quaternary ammonium compounds; buffering ingredients such as alkali metal chloride; antioxidants such as sodium metabisulfite; and other conventional ingredients such as sorbitan monolaurate.

Combinations with Other Therapeutic Agents

In the methods and uses of the present invention the combinations of agents of the invention can also be administered concomitantly with other therapeutic agents. In an embodiment, the present invention provides a method of preventing or treating pain that includes concomitantly administering to a subject in need thereof an effective amount of a first therapeutic agent comprising the combinations and compositions of the invention, and a second therapeutic agent. For example, the second therapeutic agent may increase the analgesic effectiveness of the agent or combination, for example by increasing the penetration of the alpha₂-adrenergic agonist, nitric oxide donor, PA inhibitor and/or PDE inhibitor.

Non-limiting examples of second therapeutic agents contemplated for use in the methods of the invention include analgesics known in the art, for example cyclooxygenase inhibitors and non-steroidal anti-inflammatory drugs (NSAIDs) such as acetyl salicylic acid, ibuprofen and naproxen, peripheral analgesic agents, and narcotic analgesics. Non-limiting examples of additional analgesics include capsaicin, lidocaine, bupivacaine, mepivacaine, ropivacaine, tetracaine, etidocaine, chloroprocaine, prilocalne, procaine, benzocaine, dibucaine, dyclonine hydrochloride, pramoxine hydrochloride and proparacaine. Other agents employed for the treatment of neuropathic pain which may be used in the methods and compositions of the invention include ketamine (an NMDA receptor antagonist), amitriptyline (a tricyclic antidepressant), gabapentin or pregabalin (α2δ calcium channel agents) and guanethidine (a sympathetic blocking agent), in combination or independently.

Concomitant administration includes co-administration (simultaneous administration of the first and second agent) and sequential administration (administration of the first agent, followed by the second agent, or administration of the second agent, followed by the first agent). The combination of agents used within the methods described herein may have a therapeutic additive or synergistic effect on the condition(s) or disease(s) targeted for treatment. The combination of agents used within the methods described herein may also reduce a detrimental effect associated with at least one of the agents when administered alone or without the other agent(s). For example, the toxicity of side effects of one agent may be attenuated by the other, thus allowing a higher dosage, improving patient compliance, or improving therapeutic outcome. Physicians may achieve the clinical benefits of previously recognized drugs while using lower dosage levels, thus minimizing adverse side effects. In addition, two agents administered simultaneously and acting on different targets may act synergistically to modify or ameliorate pain and/or disease progression or symptoms.

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention.

### Materials and Methods

### Animals

Male Long Evans rats (225-250 g, Charles River, St. Constant, QC) arrived 7 days before experiments. SPARC-null mice (20-25 g) were bred at the Benaroya Research Institute at Virginal Mason, Seattle, Wash. and transported to McGill University. Methods were approved by the Animal Care committee of McGill University, and conformed to ethical guidelines of the Canadian Council on Animal Care and the International Association for the Study of Pain.

### Drugs

Drugs used included sodium pentobarbital (Ceva Sante Animal, Libourne, France), lisofylline (Cayman Chemical, Ann Arbor, Mich.), linsidomine (Tocris, Ellisville, Mo.), apraclonidine, pentoxifylline and S-nitroso-N-acetyl-penicillamine (SNAP) (all obtained from Sigma-Aldrich, St. Louis, Mo.). All ingredients (see below) for the ointment base were purchased from Sigma-Aldrich, St. Louis, Mo.

### Analgesic Formulations

Ointment type analgesic formulations containing the above-mentioned drugs were prepared according to standard pharmaceutical procedures. The ointments were formulated using a composite, water-soluble polyethylene glycol base system employing carbowax (PEG 3350) and PEG 400 in the ratio of 60:40 respectively. The water-soluble base system was selected due to its non-sticky nature. Briefly, the required amounts of the active ingredients were first weighed out and then added to the already molten base in decreasing order of their melting points, and stirred well. After uniform melting, the formulation was brought to room temperature to ensure proper solidification. A standard amount of 150 mg (mean±SEM=150.88+2.7 mg) of the ointment was used for all rat hind paw applications throughout the experimental procedure, and half this amount for mouse hind paw application.

### Induction of Ischemia-Reperfusion (IR) Injury in the Rat Hind Paw

Chronic post-ischemia pain (CPIP) was generated following exposure to prolonged hind paw ischemia and reperfusion (Coderre et al., Pain, 112(1):94-105, 2004). Briefly, rats were anesthetized over a 3 h period with a bolus (55 mg/kg, i.p.) followed by chronic i.p. infusion of sodium pentobarbital for 2 h at a rate of 0.15 mL/h. After induction of anesthesia, a Nitrile 70 Durometer O-ring (O-rings West, Seattle, Wash.) with 5.5 mm internal diameter was placed around the rat's left hind limb proximal to the ankle joint. The tight-fitting O-rings produce a complete blockade of arterial blood flow as measured using laser Doppler flowmetry. The ring was then left in place for 3 h. The termination of sodium pentobarbital anesthesia was timed so that the rats recovered fully within 30-60 min following reperfusion. Rats were tested between 2 and 14 days post-IR injury.

### Induction of Chronic Constriction Injury (CCI)

Unilateral mononeuropathy was produced in rats using chronic constriction injury (CCI) of the sciatic nerve, as described by Bennett and Xie (Pain 33(1):87-107, 1988). Briefly, male Long Evans rats were anesthetized with an intraperitoneal dose of sodium pentobarbital (55 mg/kg) with additional doses of the anesthetic given as needed. Under aseptic conditions, a 3 cm incision was made on the lateral aspect of the left hind limb. The common left sciatic nerve was exposed by blunt dissection just above the trifurcation point and four loose ligatures were then made with 4-0 chromic catgut around the nerve with about 1 mm spacing in between. The wound was then closed in layers with 3-0 silk thread. The animals were then transferred to their home cages and left to recover. Rats were tested between 7 and 14 days post-CCI.

### Antinociceptive Testing Using SPARC-Null Mice

SPARC (secreted protein, acidic, and rich in cysteine) is a matricellular protein that is present in the intervertebral disc and in humans, levels of SPARC decrease with aging and disc degeneration. Targeted deletion of the SPARC gene has been reported to result in accelerated disc degeneration in the aging mouse (Gruber et al., J. Histochem. Cytochem. 53(9):1131-1138, 2005). Signs of extensive disc degeneration observed between 6 and 24 months of life in SPARC-null mice include decreased proteoglycan content, cell loss, and irregular collagen fibrils. Because degenerative disc disease (DDD) is one of the most common causes of chronic low back pain, this knockout model was used to assess the antinociceptive activity of the topical ointment formulations against low back pain, specifically cold allodynia, the major symptom that is referred to the hind paws with degenerating discs. A group of 10-month old mice was used for the experiments.

### Induction of Referred Muscle Pain

Referred muscle pain was induced in rats by administering 2 injections of 100 μl of acidic saline (pH 4.0) intramuscularly into the gastrocnemius muscle with the second injection five days following the first (Sluka et al., Muscle Nerve 24(1):37-46, 2001). Rats were tested for hind paw mechanical allodynia 24 h after the second injection.

### Induction of Inflammatory Pain

Inflammatory pain was induced in rats by injecting 50 μl of 1 mg/ml complete Freund's adjuvant (CFA) into the plantar hind paw (Ladarola et al., Pain 35(3), 313-326, 1988). Rats were tested for hind paw mechanical allodynia 48 h after the CFA injection.

### Mechanical Sensitivity Testing

The plantar surface of the ipsilateral hind paw was tested for mechanical allodynia in CPIP and CCI rats. Nylon monofilaments were applied in either ascending (after negative response) or descending (after positive response) force as necessary to determine the filament closest to the threshold of response. Each filament was applied for 10 s or until a flexion reflex occurred. The minimum stimulus intensity was 1 g and the maximum was 15 g. Based on the response pattern, and the force of the final filament (5th stimulus after first direction change), the 50% threshold (grams) was calculated as (10[Xf+kδ])/10000 where Xf=filament number of the final von Frey hair used, k=value for the pattern of positive/negative responses and δ=mean difference in log unit between stimuli (here, δ=0.220, for more details see Chaplan et al., J. Neurosci. Methods, 53(1):55-63, 1994). Mechanical sensitivities were assessed prior to and following several pharmacological treatments.

### Cold Sensitivity Testing

Cold sensitivity was assessed in SPARC-null mice by measurement of the total time spent in acetone-evoked behaviours over 1 minute after a drop (25 μL) of acetone was applied gently to the plantar surface of the foot (behaviours=paw elevation, flinching, biting, licking, and scratching time).

### Measurement of Blood Flow and Post-Occlusive Reactive Hyperemia

Plantar blood flow and post-occlusive reactive hyperemia were measured using a laser Doppler perfusion and temperature monitor (DRT4, Moor Instruments, Wilmington, Del.) to assess microvascular function (Morales et al., Microvasc. Res., 69(1-2):17-23, 2005). Briefly, rats (n=6 for each group) were anesthetized with bolus (1 g/kg) and maintenance doses (200 mg/kg prn) of urethane, and placed in the prone position. Body temperature was monitored through a rectal thermometer coupled to a heating pad (FHC, Bowdoinham, Me.), and was maintained between 37.5° C. and 39.0° C. throughout the experiment. The plantar blood flow of the ipsilateral hind paw was monitored using a laser-emitting probe placed in between the tori pads of the hind paw, along the midline. Prior to recording responses to occlusion, each rat underwent a period of stabilization lasting 20 to 30 minutes. At the rate of one sample per second, an initial 10 minute baseline blood flux was recorded, followed by a 2 minute occlusion induced by pressurizing and inflating a tight fitting loop of Tygon® R 3603 tubing (outside diameter=2.38 mm, inside diameter=0.79 mm, wall thickness=0.79 mm) connected to an air-filled pump-driven 60 mL syringe (Model 11, Harvard Apparatus, Montreal, QC), creating a tourniquet around the ankle joint. Ischemia was confirmed by observing a flux reduction greater than 95% of pre-occlusion value. Two minutes later, the pressure was released quickly and the tourniquet loosened to allow reperfusion to occur. Post-occlusive reactive hyperemia was monitored by continuously sampling flux at the rate of 1/sec for 2 min following the onset of reperfusion. PTX (25 mg/kg, i.p.) was administered ten minutes post-reperfusion. Beginning 20 minutes after drug administration, a second 2 min occlusion was induced by the above procedure, and was also followed by a 2 min recording of post-occlusive reactive hyperemia at the rate of 1 sample/sec.

### Near Infrared Spectroscopy (NIRS) in Human CRPS-I Patients

These studies were performed to confirm the validity of our animal model of CRPS-I, and to determine whether microvascular dysfunction and poor muscle oxygenation contribute to the pathology of CRPS-I in human patients. MRS sensors for muscle oxygenation (NIRO 200, Hamamatsu Photonics, Japan) were fixed either on the anterior aspect of the forearm (arm CRPS-I) or over the thenar eminence of the hand (hand CRPS-I). The NIRO 200 provides continuous, non-invasive monitoring of the relative concentration changes in oxygenated hemoglobin (HbO₂), deoxygenated hemoglobin (HHb) and total hemoglobin (Hb) and myoglobin (Mb) following the absorption of light at different wavelengths (775, 810, 850 and 910 nm). Tissue oxygenation index was then calculated to determine muscle oxygenation [TOI=HbO₂/(HbO₂+HHb)×100, expressed in %]. Muscle oxygenation recordings were taken continuously, and plotted in real time over a 3 min baseline period.

Following basal NIRS measurement, in one study we assessed muscle oxygenation during a 2 min exercise period, and for 3 min post-exercise for hands of a CRPS-I patient and a gender matched control. For the exercise period, the CRPS-I patient and control subject performed dynamic hand-grip exercise using a squeeze dynanometer (Samon Preston, Toronto, ON) at 20% Maximal Voluntary Contraction (1 repetition per sec) for 2 min. In a separate session, a CRPS-I patient followed the above procedures except that the NIRS recording during exercise and post-exercise periods were replaced by recording during ischemia and reactive hyperemia periods. Ischemia was induced using a blood pressure cuff at the upper arm (above the painful region for the affected arm). After 3 mins of baseline, rapid arterial occlusion of the unaffected arm was provoked by inflation of the cuff for a further 2 min at 50 mm Hg above the resting systolic pressure. The cuff was then suddenly deflated to cause reperfusion. Muscle saturation measurements were taken at the forearm both during the ischemia period and for 3 min after reperfusion during reactive hyperemia. This procedure, including baseline measurement, was then repeated for the CRPS-I affected limb.

### Pharmacological Treatment

### CPIP

The topical formulations containing a nitric oxide donor, an α₂-adrenergic agonist or a phosphatidic acid inhibitor were tested for their anti-allodynic effects either singly or in combination with each other in definite proportions at concentrations for single agents selected from the published literature in different animal models of pain. Accordingly, the rats received 150 mg of the respective ointment with the first half on the plantar aspect of their hind paws followed by the second half applied on the dorsal surface; in both cases by uniform gentle application using fingers. The rats were monitored immediately after application to make sure they did not lick their paws.

In the single drug pharmacological trials, pentoxifylline was tested at 0.6, 1.2, 2.5 and 5% W/W, clonidine at 0.0075, 0.015, 0.03 and 0.06% W/W, linsidomine at 0.2, 0.4, 0.8 and 1.6% W/W, SNAP at 0.0625, 0.125, 0.25 and 0.5% W/W, lisofylline at 0.0625, 0.09, 0.125 and 0.25% W/W and apraclonidine at 0.005, 0.01, 0.02 and 0.04% W/W.

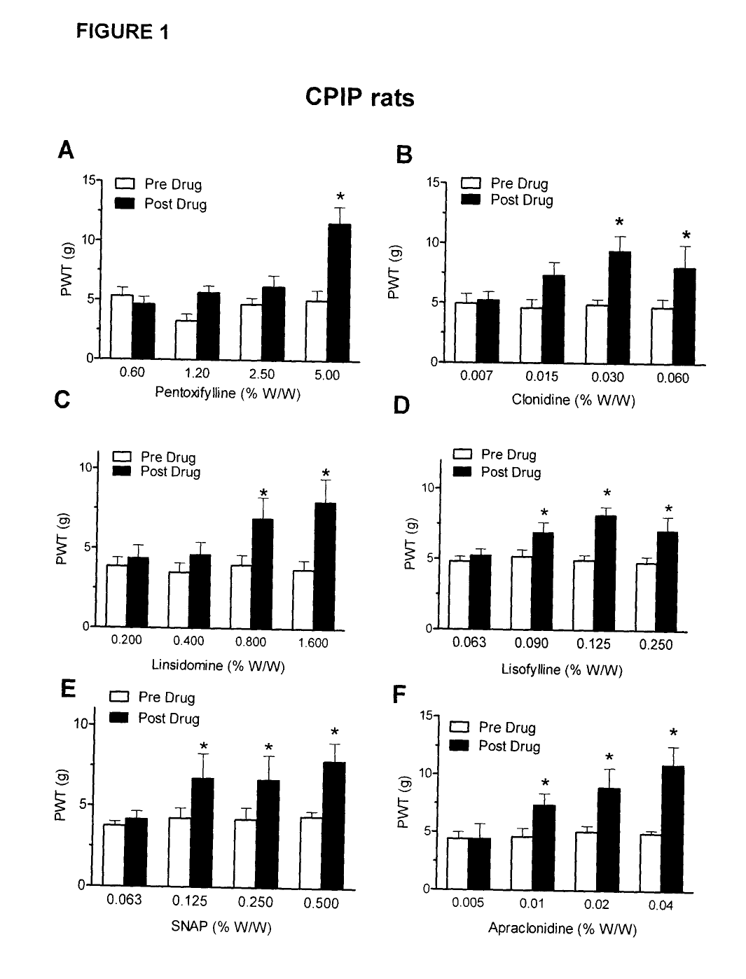

In separate groups of rats, formulations containing pentoxifylline or lisofylline were tested in specific drug combinations with constant percentage amounts of clonidine, linsidomine, SNAP or apraclonidine. The selected concentrations of these agents were all observed to be inactive when tested in the single drug trials (FIG. 1). Accordingly, the formulations tested in the combination trials included clonidine (0.0075% W/W) with pentoxifylline (0.3, 0.6 and 1.2% WAN), linsidomine (0.4% W/W) with pentoxifylline (0.075, 0.15 and 0.3% W/W), linsidomine (0.4% W/W) with lisofylline (0.03175, 0.0625 and 0.075% W/W), SNAP (0.0625% W/W) with lisofylline (0.008, 0.015, 0.033 and 0.063% W/W) and apraclonidine (0.005% W/W) with lisofylline (0.0078, 0.0156 and 0.0313% W/W). A third cohort of rats was used to confirm the local action of the tested formulations. For this study, the formulations were applied to the contralateral paw and the ipsilateral paw was tested for anti-allodynic effects. In addition, vehicle (ointment base) application to the ipsilateral paw was also evaluated. The most effective drug combinations were tested in this manner. Accordingly, the combinations included pentoxifylline (0.6% W/W) and clonidine (0.0075% W/W), pentoxifylline (0.3% W/W) and linsidomine (0.4% W/W), lisofylline (0.09% W/W) and linsidomine (0.4% W/W), lisofylline (0.0625% WAN) and SNAP (0.0625% W/W) and lisofylline (0.03125% W/W) and apraclonidine (0.005% W/W). All the rats underwent initial baseline assessment before application of the ointment followed by testing at 45 minutes post-application.

### CCI

In rats that underwent CCI, the most effective drug combinations previously determined from CPIP experiments were tested for their anti-allodynic effects following either ipsilateral or contralateral hind paw application. The combinations included pentoxifylline (0.6% W/W) and clonidine (0.0075% W/W), pentoxifylline (0.3% W/W) and linsidomine (0.4% W/W), lisofylline (0.09% W/W) and linsidomine (0.4% W/W), lisofylline (0.0625% W/W) and SNAP (0.0625% W/W) and lisofylline (0.03125% W/W) and apraclonidine (0.005% WAN). All of the rats underwent initial baseline assessment before application of the ointment followed by testing at 45 minutes post-application.

### SPARC Knock-Out Model of Low Back Pain

A single drug combination was tested in a cohort of 10 month-old

SPARC-null mice for its effect against cold allodynia. The combination tested was apraclonidine (0.005% W/W) with lisofylline (0.03% W/W). All of the mice underwent an initial baseline assessment before application of the ointment followed by testing at 15 and 45 minutes post-application. A vehicle group of six mice was run alongside to see possible drug effects.

### Referred Muscle Pain

A single drug combination (with two doses) was tested in rats with referred muscle pain. The combination tested was linsidomine (0.4% W/W) with either 0.15% W/W or 0.4% W/W pentoxifyllline. All of the rats underwent an initial pre-drug assessment before application of the ointment followed by testing at 60 minutes post-application.

### Inflammatory Pain

A single drug combination (with two doses) was tested in rats with inflammatory pain. The combination tested was linsidomine (0.4% W/W) with either 0.15% W/W or 0.4% W/W pentoxifylline. All of the rats underwent an initial pre-drug assessment before application of the ointment followed by testing at 45 minutes post-application.

### Data Analysis

Von Frey thresholds and mean blood flow values during post-occlusive reactive hyperemia were averaged by group and/or treatment and subjected to analysis of variance (ANOVA) using repeated measures. Pair-wise comparisons of group means were performed using Fisher's LSD tests after the observation of significant effects of drug treatment. The total duration of acetone-induced behaviours was measured in seconds, averaged by group and treatment time and subjected to repeated measures ANOVA followed by post hoc Fisher's LSD tests.

Shifts in drug anti-allodynic potency obtained by the use of combination treatments were illustrated by first calculating the difference between post and pre drug measures for each rat, then averaging these differences by treatment group. Mean differences were then plotted on a semilog scale of the amount of drug used per application.

### Results

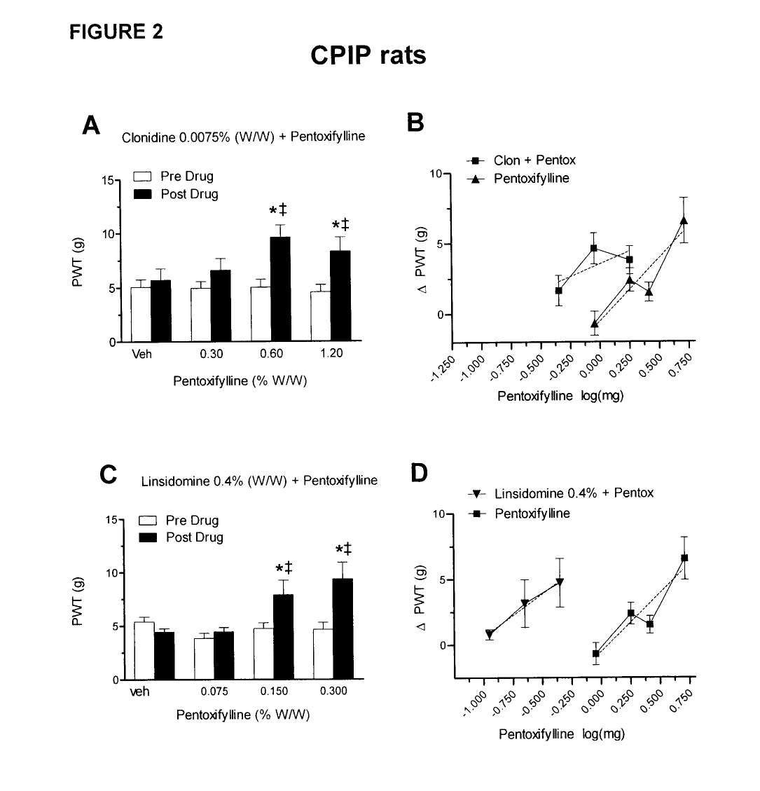

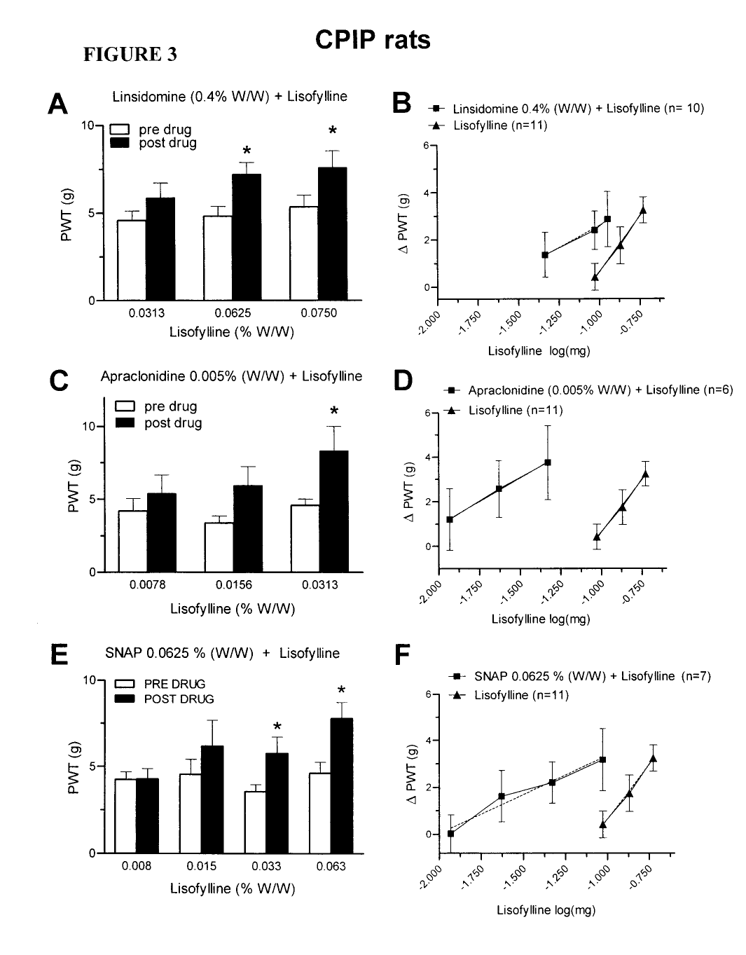

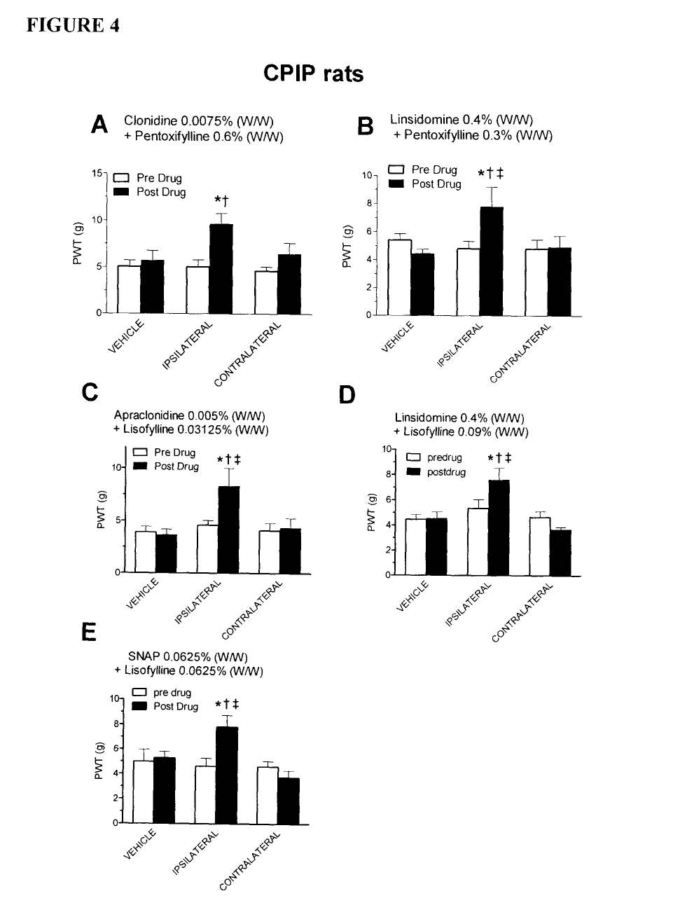

We report herein the effects of combinations of either apraclonidine or clonidine (alpha₂-adrenergic agonists), or of linsidomine or SNAP (nitric oxide donors) with pentoxifylline or lisofylline (PDE/PA inhibitors) in a rat model of CRPS-I. We found that for each of the 6 agents, topical hind paw application produces significant anti-allodynic effects in our rat model of CRPS-I (FIG. 1). Combination of a single low dose of either clonidine (FIGS. 2A,B) or linsidomine (FIGS. 2C,D) with pentoxifylline produces significant anti-allodynic effects, and shifts the pentoxifylline dose-response curve to the left producing synergistic anti-allodynic effects. Also, combination of a single low dose of linsidomine (FIGS. 3A,B), apraclonidine (FIGS. 3C,D) or SNAP (FIGS. 3E,F) with lisofylline produces significant anti-allodynic effects, and shifts the lisofylline dose-response curve to the left producing synergistic anti-allodynic effects. We believe these are local effects, since topical application of 5 of these combinations to the contralateral hind paw did not reduce allodynia in the injured hind paw (FIG. 4A-E), despite significant effects when given to the ipsilateral hind paw.

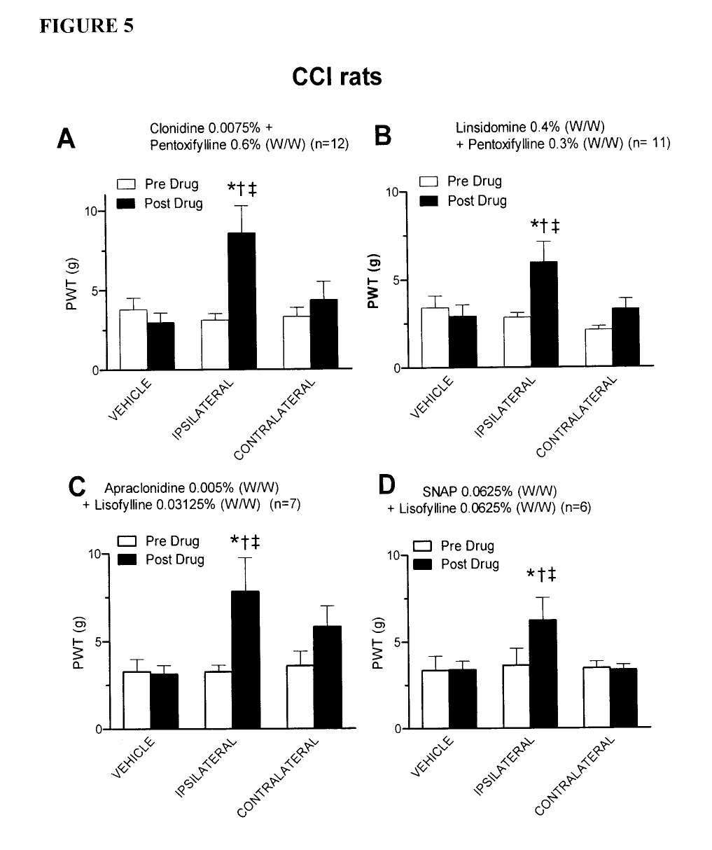

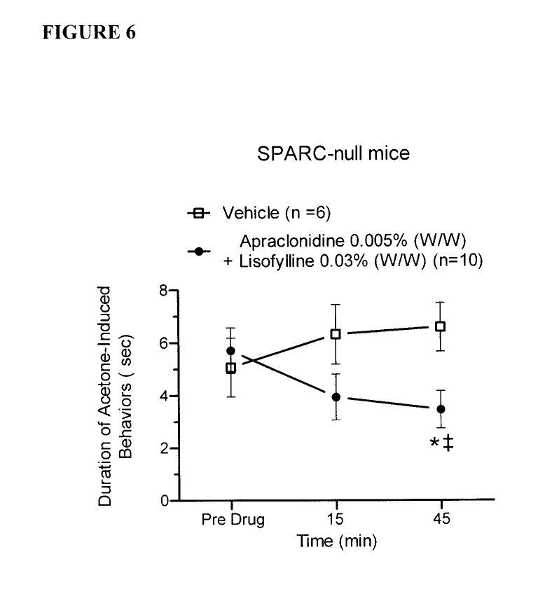

We have also shown that the low dose combinations of four of these combinations are able to reduce allodynia in neuropathic rats (FIG. 5A-D), again based on a local effect. In addition, one of the combinations reduced cold allodynia in the hind paws of SPARC-null mice (FIG. 6). SPARC-null mice have degenerative disc disease leading to back pain with referred cold allodynia in the hind paws. Importantly, the topical combinations are produced with very low doses that do not appear to produce any systemic side-effects, and were able to reduce established pain in these three animal models.

### Mechanical Allodynia

### I. Single Drug Trials in CPIP Rats

Pentoxifylline significantly attenuated mechanical allodynia when tested at 5% W/W (FIG. 1A). FIG. 1B shows the dose response profile for clonidine wherein significant anti-allodynic effects were observed at 0.03 and 0.06% W/W. Linsidomine was tested at four different concentrations, of which 0.8 and 1.6% W/W were observed to be significantly different from their pre-drug values (FIG. 1C). Lisofylline was tested at four different concentrations, of which only the lowest concentration (0.063% W/W) was observed to be inactive (FIG. 1D). FIG. 1E shows the dose response profile for SNAP, wherein significant effects were observed for all the concentrations tested except the lowest one. Similarly, apraclonidine was observed to be effective against mechanical allodynia at all concentrations except the lowest one (FIG. 1F).

### II. Combination Drug Trials in CPIP Rats

i. Combinations with Pentoxifylline

In the first combination trial, clonidine was kept constant throughout the experiment and the concentration of pentoxifylline was varied to determine if there was any increase in overall potency of the formulation. From FIG. 2A, it is very clear that pentoxifylline showed significant anti-allodynic effects at 0.6 and 1.2% W/W when combined with 0.0075% W/W of clonidine. In particular, 0.6 and 1.2% W/W of pentoxifylline were devoid of any effect when tested singly. The increase in potency of the formulation due to the addition of clonidine is very much evident from the leftward shift of the combination regression line from that of the single drug response (FIG. 2B).

In the second combination trial, linsidomine was kept constant at 0.4% W/W and the concentration of pentoxifylline was varied throughout the experiment. FIG. 2C shows the dose response profile of the combination wherein significant anti-allodynic effects were observed for the combination at 0.15 and 0.3% W/W of pentoxifylline. Moreover, combination with linsidomine reduces the net requirement of pentoxifylline to produce the same degree of anti-allodynic effect. This is very much evident from FIG. 2D, which clearly shows a leftward shift of the combination regression line from that of the single drug response.

ii. Combinations with Lisofylline

Three combination trials were conducted keeping linsidomine, SNAP or apraclonidine constant throughout the experiment and varying lisofylline concentration in each of the combinations.

In the first trial, linsidomine was kept constant at 0.4% W/W and the anti-allodynic effects were assessed using different concentrations of lisofylline. FIG. 3A shows the dose response profile of the combination. From the profile, it is clear that 0.0625% W/W lisofylline (inactive when tested singly) exhibited a significant anti-allodynic effect when combined with linsidomine. FIG. 3B shows the leftward shift in the dose response profile of the combination with respect to the single drug response. The combination with apraclonidine proved to be even more effective than that with linsidomine in that lisofylline when tested at 0.0313% W/W showed a significant anti-allodynic effect. FIG. 3D shows the dose response profile shift to the left from that of the single dose response. Similarly, the combination with SNAP also proved to be very effective, wherein significant anti-allodynic effects were observed when tested at 0.033 and 0.063% W/W of lisofylline. FIG. 3F shows the leftward shift in the combination dose response profile with respect to that of single drug treatment.

iii. Contralateral and Vehicle Control Trials in CPIP Rats

In order to confirm the local action of the formulations, control studies were undertaken wherein the ointment was applied on to the contralateral paw and the ipsilateral paw was tested for the presence of mechanical allodynia. FIG. 4 shows the response of the ipsilateral paw following application of the ointment to the contralateral paw, with ipsilateral application shown for comparison, as well as the effects of vehicle treatment. As is evident, there was no contralateral effect for all of the tested formulations at the same dose that produced significant ipsilateral effects. Hence, the results confirm that the anti-allodynic effects of the formulations are mediated locally. Furthermore, the similarly ineffectual vehicle application shows that the effects of ipsilateral ointment application are the result of drug action.

### III. Combination Drug Trials in CCI Rats

The most effective drug combinations observed in CPIP experiments were tested in CCI rats for their anti-allodynic effects, and the results are shown in FIG. 5. Again both ipsilateral and contralateral application is shown for comparison, as well as the effects of vehicle treatment. All of the tested combinations exhibited significant anti-allodynic effects both with respect to pre-drug measures and vehicle treatment following ipsilateral treatment. A contralateral control study undertaken for each combination also showed that the effects were mediated locally, as the same doses administered contralaterally did not have anti-allodynic effects.

### IV. Studies of Cold Allodynia in SPARC-Null Mice

A single formulation containing apraclonidine (0.005% W/W) and lisofylline (0.03% W/W) was tested in SPARC-null mice for its effect against cold allodynia, the predominant symptom in these mice. FIG. 6 shows the effect of the formulation in reducing the duration of acetone-induced nociceptive behaviours with respect to vehicle treatment. The effect was observed to be significantly different from vehicle control and pre-drug baseline measurement at 45 min post-application.

V. Combination Drug Trials in Rats with Referred Muscle Pain

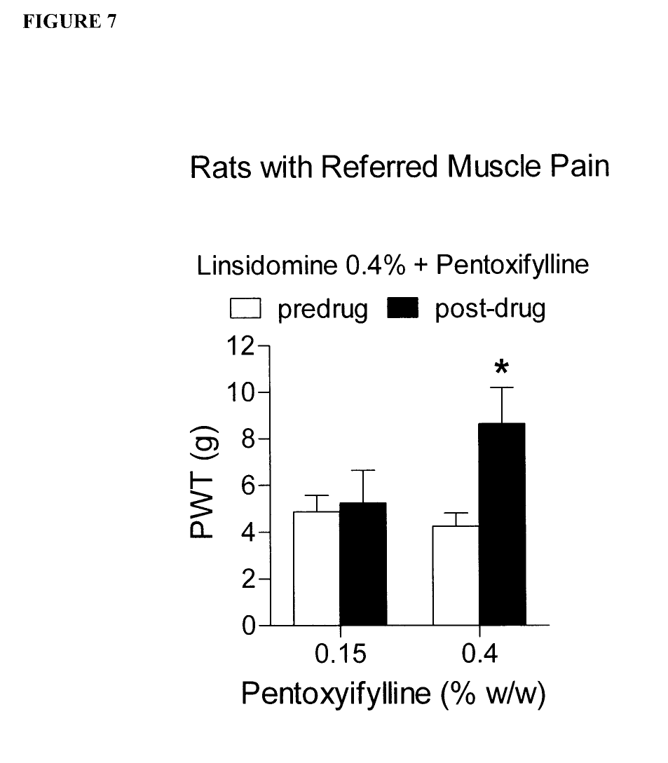

Formulations containing linsidomine (0.4% W/W) and pentoxifylline (0.15 or 0.4% W/W) were tested in rats with referred muscle pain for their effects against mechanical allodynia in the hind paw. FIG. 7 shows the effects of these formulations, with the formulation using the higher pentoxifylline dose significantly reducing mechanical allodynia, while the lower dose or vehicle had no effect.

VI. Combination Drug Trials in Rats with Inflammatory Pain

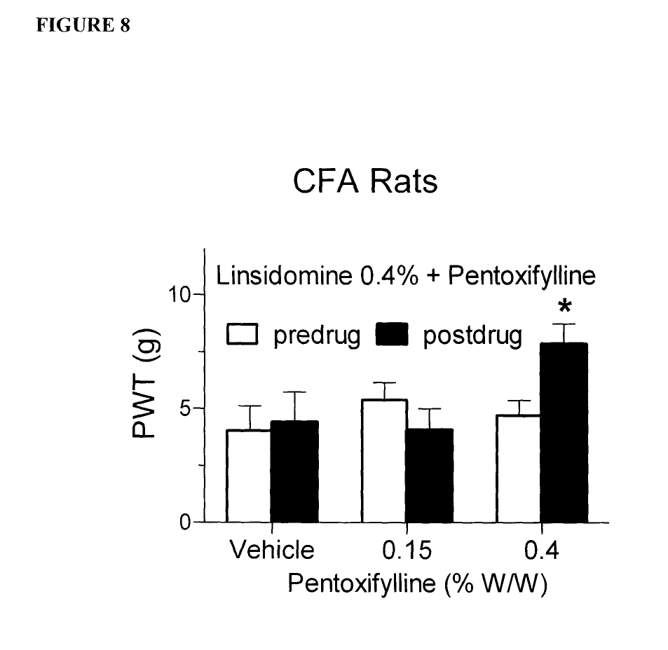

Formulations containing linsidomine (0.4% W/W) and pentoxifylline (0.15 or 0.4% W/W) were also tested in rats with inflammatory pain for their effects against mechanical allodynia in the inflamed hind paw. FIG. 8 shows the effects of these formulations, with the formulation using the higher pentoxifylline dose significantly reducing mechanical allodynia, while the lower dose or vehicle had no effect.

### VII. Effects of Systemically Administered Pentoxifylline on Both Microvascular Function and Allodynia

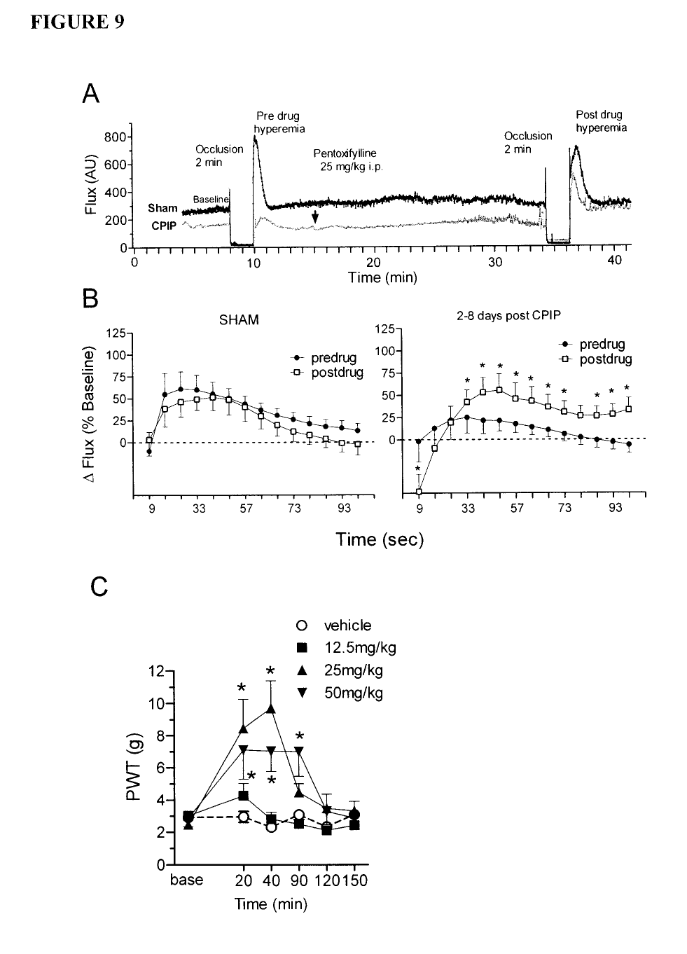

To determine whether the anti-allodynic effects of pentoxifylline are paralleled by improved microvascular function, we compared its effects on PWTs and laser Doppler measurement of hind blood flow and post-occlusive reactive hyperemia. FIG. 9A shows a representative record of basal blood flow and post-occlusive reactive hyperemia for both a sham and a CPIP rat, with the CPIP rat exhibiting a delayed post-occlusive reactive hyperemia (indicating the presence of microvascular dysfunction). FIG. 9B shows the mean blood flow during the post-occlusion hyperaemic period for groups of sham or CPIP rats that received either 25 mg/kg pentoxifylline or vehicle. CPIP rats given vehicle showed a delayed post-occlusive reactive hyperemia (i.e., microvascular dysfunction), that was reversed by treatment with pentoxifylline. In contrast, pentoxifylline had no effect on the normal post-occlusive reactive hyperemia in sham rats. FIG. 9C shows that both 25 and 50 mg/kg of pentoxifylline attenuated allodynia in CPIP rats. Thus, a dose of pentoxifylline that alleviates microvascular dysfunction, also attenuates mechanical allodynia in CPIP rats.

### VIII. Near Infrared Spectroscopic (NIRS) Assessment of Microvascular Function and Muscle Oxygenation in CRPS-I Patients

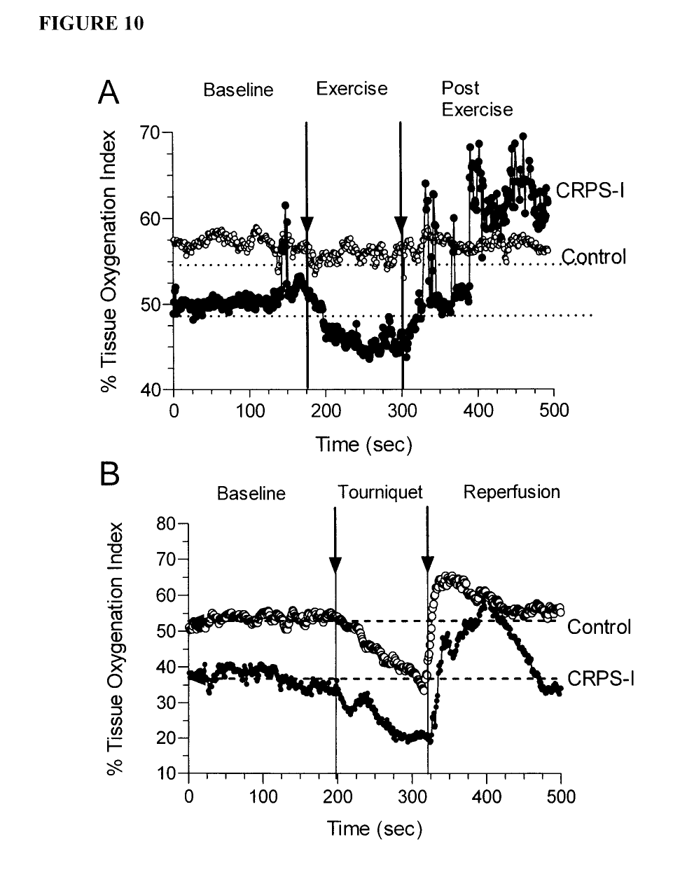

To determine the validity of our animal model of CRPS-I for the human pain condition, we used NIRS to determine whether there is similar microvascular dysfunction that leads to poor muscle oxygenation in CRPS-I patients. FIG. 10 shows the tissue oxygenation index (TOI) recordings using NIRS in the affected limb of two CRPS-I patients (closed circles) and in contralateral or healthy control limbs (open circles). A) shows Palmar TOI before exercise, during exercise, and post-exercise in a CRPS-I patient and a gender-matched control subject. Basal TOI in the CRPS-I affected hand was lower (˜7%) than the healthy subject control hand, and dropped by a further 7% during exercise. After exercise, TOI was elevated reflecting an abnormal hyperoxygenation in the CRPS-I hand. TOI remained stable throughout exercise and post-exercise in the healthy control hand. B) shows basal forearm TOI before ischemia, during ischemia (tourniquet), and post-ischemia in the affected and contralateral arms of a CRPS-I patient. Basal forearm TOI in the affected CRPS-I arm was initially ˜15% lower than the contralateral arm, and dropped another 15% during ischemia. There was a normal rapid reactive hyperoxygenation in the healthy contralateral arm, and this effect was abnormally delayed in the CRPS-I arm, reflecting microvascular dysfunction. TOI reflects the percentage of oxygenated relative to deoxygenated hemoglobin/myoglobin in the muscle beneath the NIRS probe.

These studies show that CRPS-I patients have a lower tissue oxygenation index in their affected limb, reflecting microvascular dysfunction and poor muscle oxygenation.

The contents of all documents and references cited herein are hereby incorporated by reference in their entirety.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A topical composition for the treatment of pain comprising a therapeutically effective amount of an alpha₂-adrenergic agonist or a nitric oxide donor and a therapeutically effective amount of a phosphatidic acid (PA) inhibitor or a phosphodiesterase (PDE) inhibitor, formulated in a pharmaceutically acceptable carrier for a topical composition.

2. The topical composition of claim 1, wherein the composition comprises an alpha₂-adrenergic agonist and a PA or a PDE inhibitor.

3. The topical composition of claim 1, wherein the composition comprises a nitric oxide donor and a PA or a PDE inhibitor.

4. The topical composition of claim 1, wherein the composition further comprises an alpha₂-adrenergic agonist and/or a nitric oxide donor and a PA inhibitor and/or a PDE inhibitor.

5. The topical composition of claim 1, wherein the alpha₂-adrenergic agonist is apraclonidine, clonidine, detomidine, dexamedetomidine, guanabenz, guanfacine, moxonidine, romifidine, tizanidine or xylazine.

6. The topical composition of claim 1, wherein the nitric oxide donor is isosorbide dinitrate, L-arginine, linsidomine, minoxidil, nicorandil, nitroglycerin, nitroprusside, nitrosoglutathione, or S-nitroso-N-acetyl-penicillamine (SNAP).

7. The topical composition of claim 1, wherein the PA inhibitor is lisofylline or pentoxifylline.

8. The topical composition of claim 1, wherein the PDE inhibitor is acetildenafil, avanafil, bucladesine, cilostamide, cilostazol, dipyridamole, enoximone, glaucine, ibudilast, icariin, inamrinone (formerly amrinone), lodenafil, luteolin, milrinone, mirodenafil, pentoxifylline, piclamilast, pimobendan, propentofylline, roflumilast, rolipram, RPL-554, sildenafil, tadalafil, udenafil, vardenafil or zardaverine.

9. The topical composition of claim 1, wherein the composition comprises clonidine and pentoxifylline; linsidomine and pentoxifylline; apraclonidine and lisofylline; linsidomine and lisofylline; or SNAP and lisofylline.

10. The topical composition of claim 1, further comprising an additional ingredient which increases the analgesic effectiveness of the composition.

11. The topical composition of claim 10, wherein the additional ingredient increases penetration of the alpha₂-adrenergic agonist, nitric oxide donor, PA inhibitor and/or PDE inhibitor, or wherein the additional ingredient is an analgesic.

12. (canceled)

13. The topical composition of claim 11, wherein the analgesic is selected from the group consisting of a cyclooxygenase inhibitor, an NSAID, an NMDA receptor antagonist, a tricyclic antidepressant, an α2δ calcium channel agent and guanethidine.

14. The topical composition of claim 1, wherein said composition is incorporated into a formulation selected from the group consisting of a cream, a lotion, a gel, an oil, an ointment, a spray, a foam, a liniment, an aerosol and a transdermal device for absorption through the skin.

15. The topical composition of claim 1, wherein the composition comprises about 0.005-0.5% W/W of apraclonidine, about 0.0075-0.1% W/W of clonidine or about 0.2-2% W/W of linsidomine, in combination with about 0.0078-0.5% W/W of lisofylline or about 0.075-5% W/W of pentoxifylline.

16. (canceled)

17. The topical composition of claim 1, wherein the pain is neuropathic, ischemic or muscle pain.

18. The topical composition of claim 17, wherein the pain is associated with diabetic neuropathy, complex regional pain syndrome (CRPS), angina, peripheral arterial disease, arthritis, inflammation, multiple sclerosis, fibromyalgia, peripheral neuropathy, chronic muscular pain, or chronic low back pain.

19-24. (canceled)

25. The topical composition of claim 1, wherein the composition increases tissue oxygenation in a subject; increases thermoregulatory and/or nutritive blood flow in a subject; has anti-oxidant, anti-cytokine, immunosuppressant and/or mitochondrial protective effects in a subject; reduces arterial vasospasms and/or capillary no-reflow in a subject; and/or has an anti-allodynic effect in a subject.

26-29. (canceled)

30. A method for treating neuropathic, ischemic or muscle pain in a subject in need thereof, comprising administering a therapeutically effective amount of the topical composition of claim 1 to the subject, such that the pain is treated.

31. (canceled)

32. The method of claim 30, comprising topical administration of a therapeutically effective amount of apraclonidine and lisofylline to the subject.

33. The method of claim 30, wherein the subject is a human.

34-40. (canceled)

41. The method of claim 30, wherein the composition is administered transdermally.
